(19) United States
(12) Patent Application Publication
(10) Pub. No.: US 2016/0315265 A1
(43) Pub. Date: Oct. 27, 2016

(54) 4,7-PHENANTHROLINE CONTAINING POLYMER AND ORGANIC ELECTRONIC DEVICE

(71) Applicant: CAMBRIDGE DISPLAY TECHNOLOGY LIMITED, Godmanchester (GB)

(72) Inventors: Jeremy Burroughes, Godmanchester (GB); Tania Zuberi, Godmanchester (GB); Rajesh Kumar Janarthanan, Coimbatore (IN); Kandasamy Shanmugasundaram, Krishnagiri (IN)

(73) Assignee: CAMBRIDGE DISPLAY TECHNOLOGY LIMITED, Godmanchester (GB)

(21) Appl. No.: 15/101,561

(22) PCT Filed: Nov. 25, 2014
(86) PCT No.: PCT/GB2014/053480

§ 371 Date: Jun. 3, 2016

(30) Foreign Application Priority Data
Dec. 3, 2013 (GB) 1321318.6

(51) Int. Cl. H01L 51/00; C09K 11/06; C09K 11/02; C08G 61/12; C07D 471/04

(52) U.S. Cl. CPC H01L 51/0035; C08G 61/122; C07D 471/04; C09K 11/025; C09K 11/06; C09K 2211/1466; H01L 51/56

(57) ABSTRACT

A polymer comprises repeat units of formula (I): wherein: R¹ independently in each occurrence is a substituent, and two substituents R1 may be linked to form a ring; R² in each occurrence independently is H or a substituent, and two substituents R2 may be linked to form a ring; R³ independently in each occurrence is a substituent; Ar¹ independently in each occurrence is an aryl or heteroaryl group that may be unsubstituted or substituted with one or more substituents; x is 0, 1 or 2; y is 0, 1 or 2; and z independently in each occurrence is 0, 1 or 2. Such repeat units have a relatively deep LUMO level that may reduce the barrier to transport of electrons from the electron-transporting layer to the light-emitting layer of an organic light emitting diode having an active layer comprising such a polymer.

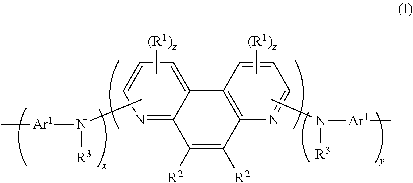

### BACKGROUND OF THE INVENTION

Electronic devices containing active organic materials are attracting increasing attention for use in devices such as organic light emitting diodes (OLEDs), organic photoresponsive devices (in particular organic photovoltaic devices and organic photosensors), organic transistors and memory array devices. Devices containing active organic materials offer benefits such as low weight, low power consumption and flexibility. Moreover, use of soluble organic materials allows use of solution processing in device manufacture, for example inkjet printing or spin-coating.

An OLED may comprise a substrate carrying an anode, a cathode and one or more organic light-emitting layers between the anode and cathode.

Holes are injected into the device through the anode and electrons are injected through the cathode during operation of the device. Holes in the highest occupied molecular orbital (HOMO) and electrons in the lowest unoccupied molecular orbital (LUMO) of a light-emitting material combine to form an exciton that releases its energy as light.

A light emitting layer may comprise a semiconducting host material and a light-emitting dopant wherein energy is transferred from the host material to the light-emitting dopant. For example, J. Appl. Phys. 65, 3610, 1989 discloses a host material doped with a fluorescent light-emitting dopant (that is, a light-emitting material in which light is emitted via decay of a singlet exciton).

Phosphorescent dopants are also known (that is, a light-emitting dopant in which light is emitted via decay of a triplet exciton).

Suitable light-emitting materials include small molecule, polymeric and dendrimeric materials. Suitable light-emitting polymers include poly(arylene vinylenes) such as poly(p-phenylene vinylenes) and polymers containing arylene repeat units, such as fluorene repeat units.

A charge-transporting layer may be provided between the light-emitting layer and an electrode.

Efficiency of an OLED may be affected by efficiency of charge transport, which in turn may be affected by the HOMO and LUMO levels of hole-transporting materials and electron-transporting materials respectively. WO 2004/041902 discloses that the LUMO level of a polyfluorene may be deepened (moved further from vacuum) by substituting the 9-position of fluorene repeat units of the polymer with electron-withdrawing groups.

US 2009/212280 discloses an OLED containing a n-doped electron-transporting layer between the light-emitting layer and the cathode of the OLED.

U.S. Pat. No. 5,393,614 discloses an OLED having an electron-transporting layer containing a compound of formula (1a):

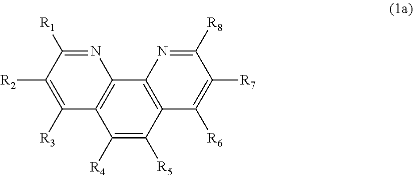

wherein R₁-R₈ independently represent a hydrogen atom, a substituted or unsubstituted alkyl group, a substituted or unsubstituted aryl group, a substituted or unsubstituted amino group, a halogen atom, a nitro group, a cyano group or a hydroxyl group.

US 2008/0154040 discloses an OLED having an electron-transporting layer containing an azafluorene compound.

### SUMMARY OF THE INVENTION

In a first aspect the invention provides a polymer comprising repeat units of formula (I):

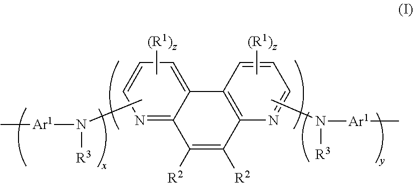

wherein:

R¹ independently in each occurrence is a substituent, and two substituents R¹ may be linked to form a ring; R² in each occurrence independently is H or a substituent, and two substituents R² may be linked to form a ring;

R³ independently in each occurrence is a substituent;

Ar¹ independently in each occurrence is an aryl or heteroaryl group that may be unsubstituted or substituted with one or more substituents;

x is 0, 1 or 2;

y is 0, 1 or 2; and

z independently in each occurrence is 0, 1 or 2.

Preferably, if x is zero y is not zero, and if y is zero x is not zero.

In a second aspect the invention provides a monomer of formula (Im):

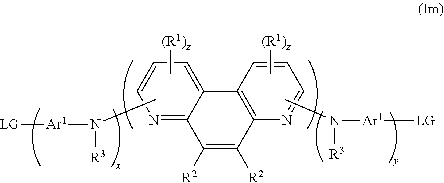

wherein LG represents a leaving group.

In a third aspect, the invention provides a method of forming a polymer according to the first aspect, the method comprising the step of polymerising a monomer according to the second aspect.

In a fourth aspect the invention provides a formulation comprising a polymer according to the first aspect and at least one solvent.

In a fifth aspect the invention provides an organic electronic device comprising an active layer comprising a polymer according the first aspect.

In a sixth aspect the invention provides a method of forming an organic electronic device according to the fifth aspect, the method comprising the step of forming the active layer.

### DESCRIPTION OF THE DRAWINGS

The invention will now be described in more detail with reference to the drawings in which:

FIG. 1 illustrates schematically an OLED according to an embodiment of the invention wherein a polymer comprising repeat units of formula (I) is provided in a light-emitting layer of the device;

FIG. 2 illustrates schematically an OLED according an embodiment of the invention wherein a polymer comprising repeat units of formula (I) is provided in a light-emitting layer of the device between an anode and an electron-transporting layer of the device;

FIG. 3A is a schematic energy level diagram illustrating the energy levels of the device of FIG. 2; and

FIG. 3B is a schematic energy level diagram illustrating the energy levels of a comparative device.

### DETAILED DESCRIPTION OF THE INVENTION

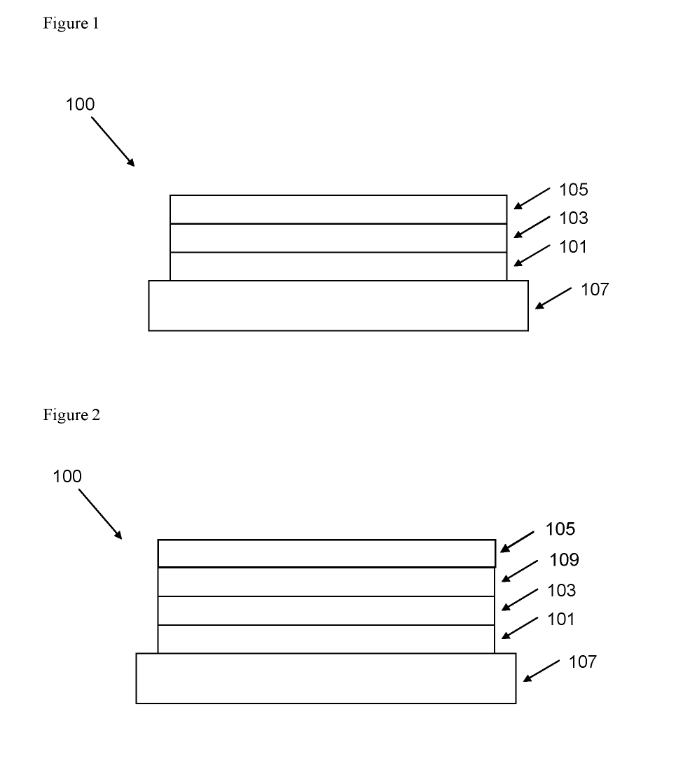

FIG. 1, which is not drawn to any scale, illustrates an OLED 100 according to an embodiment of the invention comprising an anode 101, a cathode 105 and a light-emitting layer 103 between the anode and cathode. The device 100 is supported on a substrate 107, for example a glass or plastic substrate.

One or more further layers may be provided between the anode 101 and cathode 105, for example hole-transporting layers, electron transporting layers, hole blocking layers and electron blocking layers. The device may contain more than one light-emitting layer.

Preferred device structures include:

Anode/Hole-injection layer/Light-emitting layer/Cathode

Anode/Hole transporting layer/Light-emitting layer/Cathode

Anode/Hole-injection layer/Hole-transporting layer/Light-emitting layer/Cathode

Anode/Hole-injection layer/Hole-transporting layer/Light-emitting layer/Electron-transporting layer/Cathode.

Preferably, an electron-transporting layer is present, more preferably a n-doped electron-transporting layer.

Preferably, at least one of a hole-transporting layer and hole injection layer is present. Preferably, both a hole injection layer and hole-transporting layer are present.

A polymer comprising a repeat unit of formula (I) may be provided in a charge-transporting layer and/or in a light-emitting layer of the device. A doped or undoped polymer comprising a repeat unit of formula (I) may be provided in an electron-transporting layer of an OLED. An electron-transporting layer comprising repeat units of formula (I) may be n-doped. Exemplary n-dopants for a polymer comprising repeat units of formula (I) include sodium napthalenide and derivatives thereof; organometalic derivatives comprising a metal atom, where the metal is a d-block and f-block element, sandwiched between two carbocyclic rings such as cobaltocene or ruthenocene, and derivatives thereof; alkali metal salts including Li₂CO₃, NaHCO₃, CsCO₃ and derivatives thereof; alkali metals; and alkali earth metals, for example lithium, sodium, potassium, rubidium, calcium, strontium and barium.

If x and y of formula (I) are each 0 then the polymer is preferably provided in a light-emitting layer of the device or an electron transporting layer of the device. If at least one of x and y of formula (I) is 1 or 2 then the polymer is preferably provided in a hole-transporting layer or light-emitting layer of the device. In a preferred embodiment, x and y are each 1.

FIG. 2, which is not drawn to any scale, illustrates a device according to an embodiment of the invention having layers as described in FIG. 1 and with an electron-transporting layer 109 between the light-emitting layer 103 and the cathode 105. The electron-transporting layer 109 may contain or consist essentially of an n-doped or undoped electron-transporting material. The electron-transporting material may be partially or fully doped. The electron-transporting material may be doped to produce a degenerate semiconductor. The electron-transporting material is preferably a polymer comprising repeat units of formula (I).

One or more further layers may be provided between the anode and cathode, as described with reference to FIG. 1.

The phenanthroline unit of the repeat unit of formula (I) may be linked to adjacent repeat units, or to groups —N(R³)Ar¹— in the case where x and/or y is 1 or 2, through any position on the pyridyl rings of the phenanthroline unit. The linking positions may be selected to control the extent of conjugation across the repeat unit of formula (I).

Optionally, the phenanthroline repeat unit is linked such that the repeat unit of formula (I) has formula (Ia):

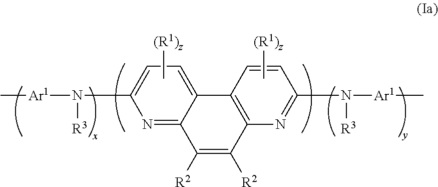

Substituents R¹ and R² may be selected from:
C₁₋₄₀ hydrocarbyl. Exemplary hydrocarbyl groups include C₁₋₂₀ alkyl; unsubstituted phenyl; phenyl substituted with one or more C₁₋₂₀ alkyl groups; a linear or branched chain of phenyl groups, wherein each phenyl may be unsubstituted or substituted with one or more substituents; and a crosslinkable group.
C₁₋₂₀ alkyl, wherein one or more non-adjacent C atoms are be replaced with optionally substituted aryl or heteroaryl, O, S, NR¹¹, C═O or —COO—, wherein R¹¹ is a substituent, optionally C₁₋₄₀ hydrocarbyl. An exemplary aryl group is phenyl, which may be unsubstituted or substituted with one or more C₁₋₂₀ alkyl or alkoxy groups.
Electron-withdrawing groups, such as groups having a positive Hammett constant. Exemplary electron withdrawing groups include C₁₋₂₀ fluoroalkyl, F, CN, and NO₂.

One or more of substituents R¹ and R² may be a crosslinkable group, for example a group comprising a double bond such and a vinyl or acrylate group, or a benzocyclobutene group.

Optionally, each occurrence of R² in formula (I) is independently selected from H and C₁₋₄₀ hydrocarbyl.

In one embodiment, each z is 0. In another embodiment, one or both z is 1 or 2

In one embodiment, x and y are each 0. In another embodiment, at least one of x and y is not 0. Repeat units wherein x and/or y are not 0 may provide hole-transporting functionality, and may be used in a hole-transporting layer and/or in a light-emitting layer.

Each Ar¹ may independently be unsubstituted or substituted with one or more substituents R⁴. Exemplary substituents R⁴ include aryl or heteroaryl that may be substituted or unsubstituted, for example phenyl that may be unsubstituted or substituted with one or more C₁₋₂₀ alkyl groups; F; CN; NO₂; and C₁₋₂₀ alkyl, wherein one or more non-adjacent C atoms may be replaced with optionally substituted aryl or heteroaryl such as phenyl that may be unsubstituted or substituted with one or more C₁₋₂₀ alkyl groups, O, S, NR¹¹, C═O or —COO— and one or more H atoms of the C₁₋₂₀ alkyl group may be replaced with F, wherein R¹¹ is a substituent, optionally C₁₋₄₀ hydrocarbyl.

Optionally, each Ar¹ is phenyl that may be 1,2-, 1,3- or 1,4-linked phenyl. Optionally, the repeat unit of formula (I) has formula (Ib):

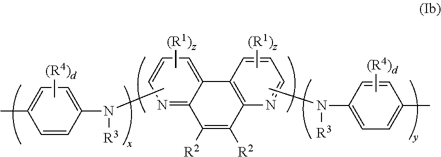

wherein R⁴ independently in each occurrence is a substituent and d independently in each occurrence is 0, 1, 2, 3 or 4.

Optionally, R³ is an aryl or heteroaryl group, optionally phenyl, which may be unsubstituted or substituted with one or more substituents. Exemplary substituents include:
substituted or unsubstituted alkyl, optionally C₁₋₂₀ alkyl, wherein one or more non-adjacent C atoms may be replaced with optionally substituted aryl or heteroaryl, O, S, NR¹¹, C═O or —COO— and one or more H atoms may be replaced with F, wherein R¹¹ is as described above; and
a crosslinkable group attached directly to the fluorene unit or spaced apart therefrom by a spacer group, for example a group comprising a double bond such and a vinyl or acrylate group, or a benzocyclobutane group.

The polymer comprising a repeat unit of formula (I) may be a homopolymer containing only repeat units of formula (I), or it may be a copolymer comprising a repeat unit of formula (I) and one or more co-repeat units other than repeat units of formula (I). In the case of a copolymer, repeat units of formula (I) may form 0.1-99 mol % of the copolymer's repeat units, optionally 1-50 mol %, 1-20 mol % or 1-10 mol %.

Exemplary co-repeat units include arylene repeat units and heteroarylene repeat units

Exemplary arylene repeat units include 1,2-, 1,3- and 1,4-phenylene repeat units, 3,6- and 2,7-linked fluorene repeat units, indenofluorene, naphthalene, anthracene and phenanthrene repeat units, and stilbene repeat units, each of which may be unsubstituted or substituted with one or more substitutents, for example one or more C₁₋₃₀hydrocarbyl substituents.

Heteroarylene repeat units are preferably electron-deficient heteroarylene units. Exemplary heteroarylene repeat units include mono- or polycyclic aromatic ring systems containing one or more N atoms and optionally one or more further heteroatoms, for example pyridine, oxadiazole, triazole, triazine and imidazole. Heteroarylene repeat units may be unsubstituted or substituted with one or more substituents R⁶ as described below.

The or each co-repeat unit is preferably provided in an amount in the range of 1-50 mol %, optionally 1-20 or 1-10 mol %.

The polymer may contain a conjugated chain of arylene repeat units.

One preferred class of arylene repeat units is phenylene repeat units, such as phenylene repeat units of formula (III):

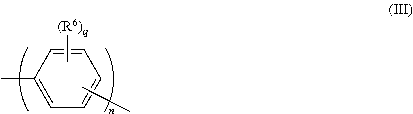

wherein q in each occurrence is independently 0, 1, 2, 3 or 4, optionally 1 or 2; n is 1, 2 or 3; and R⁶ independently in each occurrence is a substituent. Where present, each R⁶ may independently be selected from the group consisting of:
alkyl, optionally C₁₋₂₀ alkyl, wherein one or more non-adjacent C atoms may be replaced with optionally substituted aryl or heteroaryl, O, S, substituted N, C═O or —COO—,
aryl and heteroaryl groups that may be unsubstituted or substituted with one or more substituents, preferably phenyl substituted with one or more C₁₋₂₀ alkyl groups;
a linear or branched chain of aryl or heteroaryl groups, each of which groups may independently be substituted, for example a group of formula -(Ar³)ᵣ wherein each Ar³ is independently an aryl or heteroaryl group and r is at least 2, preferably a branched or linear chain of phenyl groups each of which may be unsubstituted or substituted with one or more C₁₋₂₀ alkyl groups;
electron-withdrawing groups, such as groups having a positive Hammett constant. Exemplary electron withdrawing groups include C₁₋₂₀ fluoroalkyl, F, CN, and NO₂; and.
a crosslinkable-group, for example a group comprising a double bond such and a vinyl or acrylate group, or a benzocyclobutane group.

In the case where R⁶ comprises an aryl or heteroaryl group, or a linear or branched chain of aryl or heteroaryl groups, the or each aryl or heteroaryl group may be substituted with one or more substituents R⁷ selected from the group consisting of:
alkyl, for example C₁₋₂₀ alkyl, wherein one or more non-adjacent C atoms may be replaced with O, S, substituted N, C═O and —COO— and one or more H atoms of the alkyl group may be replaced with F;
NR⁹₂, OR⁹, SR⁹, SiR⁹₃ and fluoro, nitro and cyano;

wherein each R⁹ is independently selected from the group consisting of alkyl, preferably C₁₋₂₀ alkyl; and aryl or heteroaryl, preferably phenyl, which may be unsubstituted or substituted with one or more C₁₋₂₀ alkyl groups.

Substituted N, where present, may be —NR⁹— wherein R⁹ is as described above.

Preferably, each R⁶, where present, is independently selected from C₁₋₄₀ hydrocarbyl, and is more preferably selected from C₁₋₂₀ alkyl; unusubstituted phenyl; phenyl substituted with one or more C₁₋₂₀ alkyl groups; a linear or branched chain of phenyl groups, wherein each phenyl may be unsubstituted or substituted with one or more substituents; and a crosslinkable group.

If n is 1 then exemplary repeat units of formula (III) include the following:

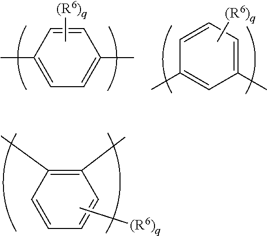

A particularly preferred repeat unit of formula (III) has formula (IIIa):

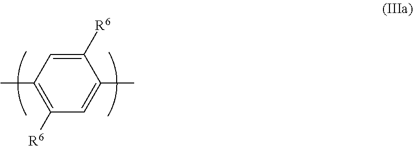

Substituents R⁶ of formula (IIIa) are adjacent to linking positions of the repeat unit, which may cause steric hindrance between the repeat unit of formula (IIIa) and adjacent repeat units, resulting in the repeat unit of formula (IIIa) twisting out of plane relative to one or both adjacent repeat units.

Exemplary repeat units where n is 2 or 3 include the following:

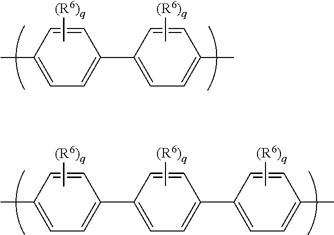

A preferred repeat unit has formula (IIIb):

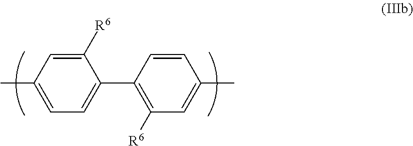

The two R⁶ groups of formula (IIIb) may cause steric hindrance between the phenyl rings they are bound to, resulting in twisting of the two phenyl rings relative to one another.

A further class of arylene repeat units are fluorene repeat units, such as repeat units of formula (IV):

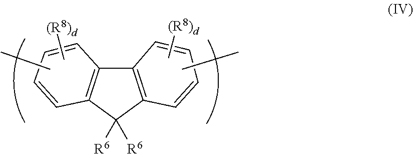

wherein R⁶ in each occurrence is the same or different and is a substituent as described with reference to formula (III), and wherein the two groups R⁶ may be linked to form a ring; R⁸ is a substituent; and d is 0, 1, 2 or 3.

The aromatic carbon atoms of the fluorene repeat unit may be unsubstituted, or may be substituted with one or more substituents R⁸. Exemplary substituents R⁸ are alkyl, for example C₁₋₂₀ alkyl, wherein one or more non-adjacent C atoms may be replaced with O, S, NH or substituted N, C═O and —COO—, optionally substituted aryl, optionally substituted heteroaryl, alkoxy, alkylthio, fluorine, cyano and arylalkyl. Particularly preferred substituents include C₁₋₂₀ alkyl and substituted or unsubstituted aryl, for example phenyl. Optional substituents for the aryl include one or more C₁₋₂₀ alkyl groups.

Substituted N, where present, may be —NR⁵— wherein R⁵ is C₁₋₂₀ alkyl; unsubstituted phenyl; or phenyl substituted with one or more C₁₋₂₀ alkyl groups.

The extent of conjugation of repeat units of formula (IV) to aryl or heteroaryl groups of adjacent repeat units may be controlled by (a) linking the repeat unit through the 3- and/or 6-positions to limit the extent of conjugation across the repeat unit, and/or (b) substituting the repeat unit with one or more substituents R⁸ in or more positions adjacent to the linking positions in order to create a twist with the adjacent repeat unit or units, for example a 2,7-linked fluorene carrying a C₁₋₂₀ alkyl substituent in one or both of the 3- and 6-positions.

The repeat unit of formula (IV) may be a 2,7-linked repeat unit of formula (IVa):

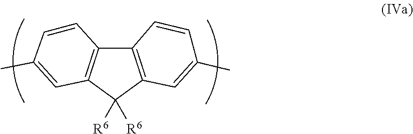

Optionally, the repeat unit of formula (IVa) is not substituted in a position adjacent to the 2- or 7-position. Linkage through the 2- and 7-positions and absence of substituents adjacent to these linking positions provides a repeat unit that is capable of providing a relatively high degree of conjugation across the repeat unit.

The repeat unit of formula (IV) may be a 3,6-linked repeat unit of formula (IVb)

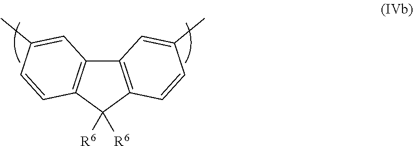

The extent of conjugation across a repeat unit of formula (IVb) may be relatively low as compared to a repeat unit of formula (IVa).

Another exemplary arylene repeat unit has formula (V):

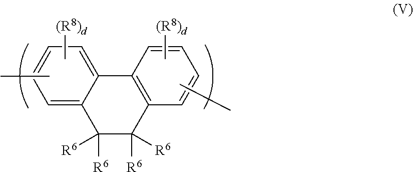

wherein R⁶, R⁸ and d are as described with reference to formula (III) and (IV) above. Any of the R⁶ groups may be linked to any other of the R⁶ groups to form an unsubstituted or substituted ring, for example a ring substituted with one or more C₁₋₄₀ hydrocarbyl groups. Aromatic carbon atoms of the repeat unit of formula (V) may be unsubstituted, or may be substituted with one or more substituents R⁸.

Repeat units of formula (V) may have formula (Va) or (Vb):

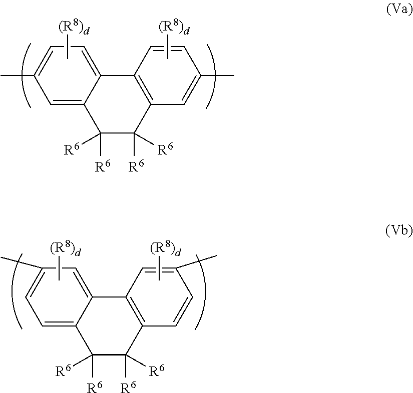

Further arylene co-repeat units include: phenanthrene repeat units; naphthalene repeat units; anthracene repeat units; and perylene repeat units. Each of these arylene repeat units may be linked to adjacent repeat units through any two of the aromatic carbon atoms of these units. Specific exemplary linkages include 9,10-anthracene; 2,6-anthracene; 1,4-naphthalene; 2,6-naphthalene; 2,7-phenanthrene; and 2,5-perylene. Each of these repeat units may be substituted or unsubstituted, for example substituted with one or more C₁₋₄₀ hydrocarbyl groups.

The polymer may contain one or more charge-transporting co-epeat units. Exemplary charge-transporting co-repeat units include repeat units of materials disclosed in, for example, Shirota and Kageyama, Chem. Rev. 2007, 107, 953-1010

Exemplary hole transporting repeat units may be repeat units of materials having a electron affinity of 2.9 eV or lower and an ionisation potential of 5.8 eV or lower, preferably 5.7 eV or lower.

Preferred hole-transporting repeat units are (hetero)arylamine repeat units, including repeat units of formula (VII):

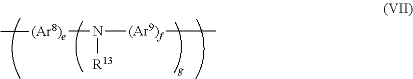

wherein Ar⁸ and Ar⁹ in each occurrence are independently selected from substituted or unsubstituted aryl or heteroaryl, g is greater than or equal to 1, preferably 1 or 2, R¹³ is H or a substituent, preferably a substituent, and e and f are each independently 1, 2 or 3.

R¹³, which may be the same or different in each occurrence when g>1, is preferably selected from the group consisting of alkyl, for example C₁₋₂₀ alkyl, Ar¹⁰, a branched or linear chain of Ar¹⁰ groups, or a crosslinkable unit that is bound directly to the N atom of formula (VII) or spaced apart therefrom by a spacer group, wherein Ar¹⁰ in each occurrence is independently optionally substituted aryl or heteroaryl. Exemplary spacer groups are C₁₋₂₀ alkyl, phenyl and phenyl-C₁₋₂₀ alkyl.

Any of Ar⁸, Ar⁹ and, if present, Ar¹⁰ in the repeat unit of Formula (VII) may be linked by a direct bond or a divalent linking atom or group to another of Ar⁸, Ar⁹ and Ar¹⁰. Preferred divalent linking atoms and groups include O, S; substituted N; and substituted C.

Any of Ar⁸, Ar⁹ and, if present, Ar¹⁰ may be substituted with one or more substituents. Exemplary substituents are substituents R¹⁰, wherein each R¹⁰ may independently be selected from the group consisting of:
substituted or unsubstituted alkyl, optionally C₁₋₂₀ alkyl, wherein one or more non-adjacent C atoms may be replaced with optionally substituted aryl or heteroaryl, O, S, substituted N, C═O or —COO— and one or more H atoms may be replaced with F; and
a crosslinkable group attached directly to the fluorene unit or spaced apart therefrom by a spacer group, for example a group comprising a double bond such and a vinyl or acrylate group, or a benzocyclobutane group

Preferred repeat units of formula (VII) have formulae 1-3:

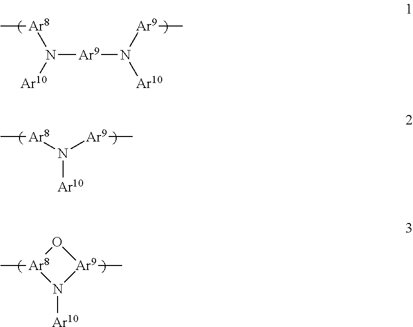

In one preferred arrangement, R¹³ is Ar¹⁰ and each of Ar⁸, Ar⁹ and Ar¹⁰ are independently and optionally substituted with one or more C₁₋₂₀ alkyl groups. Ar⁸, Ar⁹ and Ar¹⁰ are preferably phenyl.

In another preferred arrangement, the central Ar⁹ group of formula (I) linked to two N atoms is a polycyclic aromatic that may be unsubstituted or substituted with one or more substituents R¹⁰. Exemplary polycyclic aromatic groups are naphthalene, perylene, anthracene and fluorene.

In another preferred arrangement, Ar⁸ and Ar⁹ are phenyl, each of which may be substituted with one or more C₁₋₂₀ alkyl groups, and R¹³ is -(Ar¹⁰)ᵣ wherein r is at least 2 and wherein the group -(Ar¹⁰)ᵣ forms a linear or branched chain of aromatic or heteroaromatic groups, for example 3,5-diphenylbenzene wherein each phenyl may be substituted with one or more C₁₋₂₀ alkyl groups. In another preferred arrangement, c, d and g are each 1 and Ar⁸ and Ar⁹ are phenyl linked by an oxygen atom to form a phenoxazine ring.

Amine repeat units may be provided in a molar amount in the range of about 0.5 mol % up to about 50 mol %, optionally about 1-25 mol %, optionally about 1-10 mol %.

The polymer may contain one, two or more different repeat units of formula (VII).

Amine repeat units may provide hole-transporting and/or light-emitting functionality. Preferred fluorescent light-emitting amine repeat units include a blue light-emitting repeat unit of formula (VIIa) and a green light-emitting repeat unit formula (VIIb):

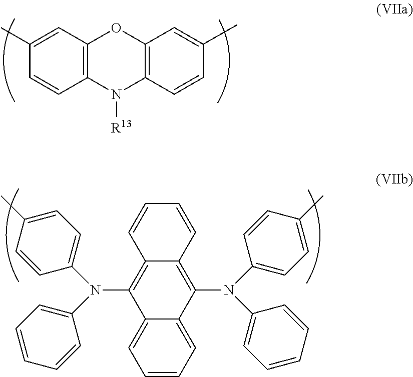

R¹³ of formula (VIIa) is preferably a hydrocarbyl, preferably C₁₋₂₀ alkyl, phenyl that is unsubstituted or substituted with one or more C₁₋₂₀ alkyl groups, or a branched or linear chain of phenyl groups wherein each said phenyl group is unsubstituted or substituted with one or more C₁₋₂₀ alkyl groups.

The repeat unit of formula (VIIb) may be unsubstituted or one or more of the rings of the repeat unit of formula (VIIb) may be substituted with one or more substituents preferably one or more C₁₋₂₀alkyl groups.

Another preferred charge-transporting repeat unit has formula (VIII):

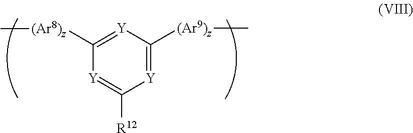

wherein Ar⁸ and Ar⁹ are as described with reference to formula (VII) above, and may each independently be substituted with one or more substituents described with reference to Ar⁸ and Ar⁹; z in each occurrence is independently at least 1, optionally 1, 2 or 3, preferably 1, R¹² is a substituent and Y is N or CR¹⁴, wherein R¹⁴ is H or a substituent, preferably H or C₁₋₁₀ alkyl. R¹² may be a C₁₋₂₀ alkyl group or a group of formula (Ar¹⁰)_{z} wherein Ar¹⁰ and z are as described above. Preferably, Ar⁸, Ar⁹ and Ar¹⁰ of formula (VIII) are each phenyl, each phenyl being optionally and independently substituted with one or more C₁₋₂₀ alkyl groups.

In one preferred embodiment, all 3 groups Y are N.

If all 3 groups Y are CR¹⁴ then at least one of Ar⁸, Ar⁹ and Ar¹⁰ is preferably a heteroaromatic group comprising N.

Each of Ar⁸, Ar⁹ and Ar¹⁰ may independently be substituted with one or more substituents. In one arrangement, Ar⁸, Ar⁹ and Ar¹⁰ are phenyl in each occurrence. Exemplary substituents include R⁵ as described above with reference to formula (V), for example C₁₋₂₀ alkyl or alkoxy.

Ar¹⁰ of formula (VIII) is preferably phenyl, and is optionally substituted with one or more C₁₋₂₀ alkyl groups or a crosslinkable unit.

Preferably, z is 1 and each of Ar⁸, Ar⁹ and Ar¹⁰ is unsubstituted phenyl or phenyl substituted with one or more C₁₋₂₀ alkyl groups.

A particularly preferred repeat unit of formula (VIII) has formula (Villa), which may be unsubstituted or substituted with one or more substituents R⁵, preferably one or more C₁₋₂₀ alkyl groups:

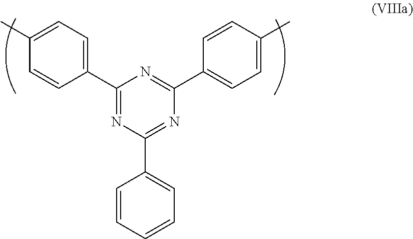

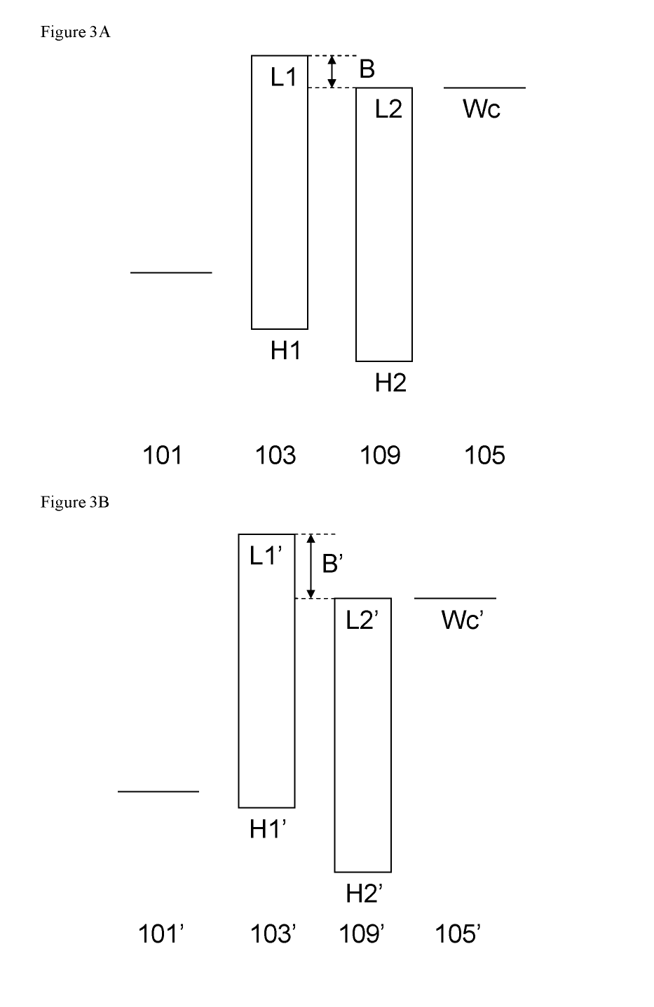

FIG. 3a, which is not drawn to any scale, illustrates HOMO and LUMO levels of materials in a device having the structure illustrated in FIG. 2.

Light-emitting layer 103 contains a polymer comprising repeat units of formula (I) having HOMO and LUMO levels H1 and L1 respectively. Adjacent electron-transporting layer 109 contains an electron-transporting material having HOMO and LUMO levels H2 and L2 respectively, although in other embodiments electron-transporting layer may contain a degenerate semiconductor.

In operation, electrons are injected from cathode 105 having a workfunction Wc into electron-transporting layer 109. Electrons are transported from electron-transporting layer into light emitting layer 103. The repeat unit of formula (I) is electron-deficient as compared to a corresponding arylene repeat unit and so the LUMO of the repeat unit is relatively deep (i.e. relatively far from vacuum level), and the barrier B to injection of electrons from the electron-transporting layer is relatively small.

Optionally, polymers comprising a repeat unit of formula (I) have a LUMO level in the range of about 2.3-2.7 eV, optionally 2.3-2.6 eV as measured by cyclic voltammetry.

For the purpose of comparison, FIG. 3b, which is not drawn to any scale, illustrates a similar device to that shown in FIG. 3a, but wherein the light-emitting layer 103′ contains a polymer containing arylene repeat units and no repeat units of formula (I). This polymer has a relatively shallow LUMO level, and the barrier B′ to injection of electrons is relatively large.

The arylene repeat units provide the polymer with a relatively shallow LUMO level, which may create a barrier to electron transport from the electron-transporting layer into the light-emitting layer.

Repeat units of formula (I) are electron-deficient as compared to arylene repeat units as a result of the nitrogen atoms of the repeat units of formula (I). Consequently, the repeat units of formula (I) have a relatively deep LUMO level that may reduce the barrier to transport of electrons from the electron-transporting layer to the light-emitting layer.

Light-emitting layer 103 may contain a polymer of the invention doped with one or more luminescent dopants. The light-emitting layer 103 may consist essentially of these materials or may contain one or more further materials, for example one or more charge-transporting materials or one or more further light-emitting materials. When used as a host material for one or more light-emitting dopants, the singlet or triplet energy level of the host material is preferably no more than 0.1 eV below that of the light-emitting material, and is more preferably about the same or higher than that of the light-emitting material in order to avoid quenching of luminescence from the light-emitting dopant.

In a preferred embodiment, light-emitting layer 103 contains a polymer of the invention and at least one of green and blue phosphorescent light-emitting materials.

A charge-transporting layer adjacent to a phosphorescent light-emitting layer preferably contains a charge-transporting material having a T₁ excited state energy level that is no more than 0.1 eV lower than, preferably the same as or higher than, the T₁ excited state energy level of the phosphorescent light-emitting material(s) of the invention in order to avoid quenching of triplet excitons migrating from the light-emitting layer into the charge-transporting layer. Accordingly, a polymer of the invention may be used as a charge-transporting material in a charge-transporting layer. In one preferred arrangement, a hole-transporting layer comprises or consists essentially of the polymer.

Triplet energy levels may be measured from the energy onset of the phosphorescence spectrum measured by low temperature phosphorescence spectroscopy (Y. V. Romaovskii et al, Physical Review Letters, 2000, 85 (5), p 1027, A. van Dijken et al, Journal of the American Chemical Society, 2004, 126, p 7718).

Polymers comprising repeat units of formula (I) are suitably amorphous polymers.

Polymer Synthesis

Preferred methods for preparation of polymers comprising repeat units of formula (I) comprise a “metal insertion” wherein the metal atom of a metal complex catalyst is inserted between an aryl or heteroaryl group and a leaving group of a monomer. Exemplary metal insertion methods are Suzuki polymerisation as described in, for example, WO 00/53656 and Yamamoto polymerisation as described in, for example, T. Yamamoto, “Electrically Conducting And Thermally Stable pi-Conjugated Poly(arylene)s Prepared by Organometallic Processes”, Progress in Polymer Science 1993, 17, 1153-1205. In the case of Yamamoto polymerisation, a nickel complex catalyst is used; in the case of Suzuki polymerisation, a palladium complex catalyst is used.

In the synthesis of a linear polymer by Yamamoto polymerisation, a monomer having two reactive halogen groups is used. According to the method of Suzuki polymerisation, at least one reactive group is a boron derivative group such as a boronic acid or boronic ester and the other reactive group is a halogen. Preferred halogens are chlorine, bromine and iodine, most preferably bromine.

In the case of a copolymer comprising repeat units of formula (I), co-repeat units may be formed in the same way. It will therefore be appreciated that repeat units illustrated throughout this application may be derived from a monomer carrying suitable leaving groups. Likewise, an end group or side group may be bound to the polymer by reaction of a suitable leaving group.

Suzuki polymerisation may be used to prepare regioregular, block and random copolymers. In particular, homopolymers or random copolymers may be prepared when one reactive group is a halogen and the other reactive group is a boron derivative group. Alternatively, block or regioregular copolymers may be prepared when both reactive groups of a first monomer are boron and both reactive groups of a second monomer are halogen.

As alternatives to halides, other leaving groups capable of participating in metal insertion include sulfonic acids and sulfonic acid esters such as tosylate, mesylate and triflate.

The base may be an organic or inorganic base. Exemplary organic bases include tetra-alkylammonium hydroxides, carbonates and bicarbonates. Exemplary inorganic bases include metal (for example alkali or alkali earth) hydroxides, carbonates and bicarbonates.

The palladium complex catalyst may be a palladium (0) or palladium (II) compound. A phosphine may be provided, either as a ligand of the palladium compound catalyst or as a separate compound added to the polymerisation mixture.

Light-Emitting Layers

An OLED may contain one or more light-emitting layers. Emission from two or more light-emitting layers may combine to produce white light.

A light-emitting layer may contain a polymer comprising repeat units of formula (I).

Suitable light-emitting materials for a light-emitting layer include polymeric, small molecule and dendritic light-emitting materials, each of which may be fluorescent or phosphorescent.

A polymer of the invention provided in a light-emitting layer may itself emit light during operation of a device, or may function as a host material for a separate light-emitting material.

Light emission from a polymer of the invention or another light-emitting material may be red, green or blue light.

A blue emitting material may have a photoluminescent spectrum with a peak in the range of 400-490 nm, optionally 420-490 nm.

A green emitting material may have a photoluminescent spectrum with a peak in the range of more than 490 nm up to 580 nm, optionally more than 490 nm up to 540 nm.

A red emitting material may optionally have a peak in its photoluminescent spectrum of more than 580 nm up to 630 nm, optionally 585-625 nm.

A light-emitting layer of an OLED may be unpatterned, or may be patterned to form discrete pixels. Each pixel may be further divided into subpixels. The light-emitting layer may contain a single light-emitting material, for example for a monochrome display or other monochrome device, or may contain materials emitting different colours, in particular red, green and blue light-emitting materials for a full-colour display.

A light-emitting layer may contain a mixture of more than one light-emitting material, for example a mixture of light-emitting materials that together provide white light emission.

A white-emitting OLED may contain a single, white-emitting layer or may contain two or more layers that emit different colours which, in combination, produce white light. The light emitted from a white-emitting OLED may have CIE x coordinate equivalent to that emitted by a black body at a temperature in the range of 2500-9000K and a CIE y coordinate within 0.05 or 0.025 of the CIE y co-ordinate of said light emitted by a black body, optionally a CIE x coordinate equivalent to that emitted by a black body at a temperature in the range of 2700-6000K.

Exemplary fluorescent polymeric light-emitting materials include polymers comprising one or more of arylene repeat units, arylene vinylene repeat units and arylamine repeat units.

Exemplary phosphorescent light-emitting materials include metal complexes comprising substituted or unsubstituted complexes of formula (IX):

ML¹_{q}L²ᵣL³ₛ (IX)

wherein M is a metal; each of L¹, L² and L³ is a coordinating group; q is a positive integer; r and s are each independently 0 or a positive integer; and the sum of (a·q)+(b·r)+(c·s) is equal to the number of coordination sites available on M, wherein a is the number of coordination sites on L¹, b is the number of coordination sites on L² and c is the number of coordination sites on L³.

Heavy elements M induce strong spin-orbit coupling to allow rapid intersystem crossing and emission from triplet or higher states. Suitable heavy metals M include d-block metals, in particular those in rows 2 and 3 i.e. elements 39 to 48 and 72 to 80, in particular ruthenium, rhodium, palladium, rhenium, osmium, iridium, platinum and gold. Iridium is particularly preferred.

Exemplary ligands L¹, L² and L³ include carbon or nitrogen donors such as porphyrin or bidentate ligands of formula (X):

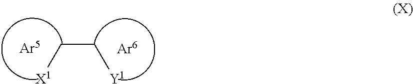

wherein Ar⁵ and Ar⁶ may be the same or different and are independently selected from substituted or unsubstituted aryl or heteroaryl; X¹ and Y¹ may be the same or different and are independently selected from carbon or nitrogen; and Ar⁵ and Ar⁶ may be fused together. Ligands wherein X¹ is carbon and Y¹ is nitrogen are preferred, in particular ligands in which Ar⁵ is a single ring or fused heteroaromatic of N and C atoms only, for example pyridyl or isoquinoline, and Ar⁶ is a single ring or fused aromatic, for example phenyl or naphthyl.

Examples of bidentate ligands are illustrated below:

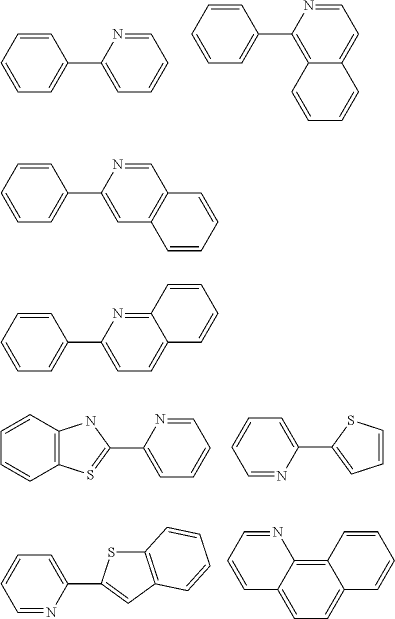

Each of Ar⁵ and Ar⁶ may carry one or more substituents. Two or more of these substituents may be linked to form a ring, for example an aromatic ring.

Other ligands suitable for use with d-block elements include diketonates, in particular acetylacetonate (acac), tetrakis-(pyrazol-1-yl)borate, 2-carboxypyridyl, triarylphosphines and pyridine, each of which may be substituted.

Exemplary substituents include groups R¹³ as described above with reference to Formula (VII). Particularly preferred substituents include fluorine or trifluoromethyl which may be used to blue-shift the emission of the complex, for example as disclosed in WO 02/45466, WO 02/44189, US 2002-117662 and US 2002-182441; alkyl or alkoxy groups, for example C₁₋₂₀ alkyl or alkoxy, which may be as disclosed in JP 2002-324679; carbazole which may be used to assist hole transport to the complex when used as an emissive material, for example as disclosed in WO 02/81448; and dendrons which may be used to obtain or enhance solution processability of the metal complex, for example as disclosed in WO 02/66552.

A light-emitting dendrimer typically comprises a light-emitting core bound to one or more dendrons, wherein each dendron comprises a branching point and two or more dendritic branches. Preferably, the dendron is at least partially conjugated, and at least one of the branching points and dendritic branches comprises an aryl or heteroaryl group, for example a phenyl group. In one arrangement, the branching point group and the branching groups are all phenyl, and each phenyl may independently be substituted with one or more substituents, for example alkyl or alkoxy.

A dendron may have optionally substituted formula (XI)

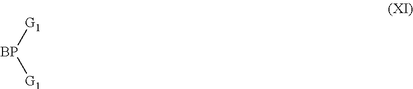

wherein BP represents a branching point for attachment to a core and G₁ represents first generation branching groups.

The dendron may be a first, second, third or higher generation dendron. G₁ may be substituted with two or more second generation branching groups G₂, and so on, as in optionally substituted formula (XIa):

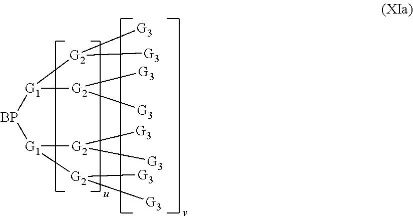

wherein u is 0 or 1; v is 0 if u is 0 or may be 0 or 1 if u is 1; BP represents a branching point for attachment to a core and G₁, G₂ and G₃ represent first, second and third generation dendron branching groups. In one preferred embodiment, each of BP and G₁, G₂ . . . Gₙ is phenyl, and each phenyl BP, G₁, G₂ . . . Gₙ₋₁ is a 3,5-linked phenyl.

A preferred dendron is a substituted or unsubstituted dendron of formula (XIb):

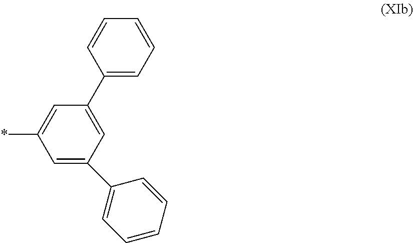 [SMILES: *c1cc(-c2ccccc2)cc(-c2ccccc2)c1]

wherein * represents an attachment point of the dendron to a core.

BP and/or any group G may be substituted with one or more substituents, for example one or more C₁₋₂₀ alkyl or alkoxy groups.

Phosphorescent light-emitting materials may be provided in a light-emitting layer with a host material. The host material may be a host polymer of the invention.

The phosphorescent light-emitting material may be physically mixed with the host polymer or may be covalently bound thereto. The phosphorescent light-emitting material may be provided in a side-chain, main chain or end-group of the polymer. Where the phosphorescent material is provided in a polymer side-chain, the phosphorescent material may be directly bound to the backbone of the polymer or spaced apart therefrom by a spacer group, for example a C₁₋₂₀ alkyl spacer group in which one or more non-adjacent C atoms may be replaced by O or S. It will therefore be appreciated that a composition of the present invention may consist of or may comprise a polymer of the invention comprising repeat units of formula (I) with a phosphorescent light-emitting material bound to the polymer.

In the case where one or more phosphorescent light-emitting materials are mixed with a host material, the phosphorescent light-emitting material(s) may make up about 0.05 wt % up to about 50 wt %, optionally about 1-40 wt % of a host/phosphorescent light-emitting material composition.

In the case where one or more phosphorescent light-emitting materials are bound to a host material, for example a host polymer, the phosphorescent light-emitting material(s) may make up about 0.01-25 mol % of the material.

Charge Transporting and Charge Blocking Layers

In the case of an OLED, a hole transporting layer may be provided between the anode and the light-emitting layer or layers. Likewise, an electron transporting layer may be provided between the cathode and the light-emitting layer or layers.

Similarly, an electron blocking layer may be provided between the anode and the light-emitting layer and a hole blocking layer may be provided between the cathode and the light-emitting layer. Transporting and blocking layers may be used in combination. Depending on its HOMO and LUMO levels, a single layer may both transport one of holes and electrons and block the other of holes and electrons.

A charge-transporting or charge-blocking layer may contain a doped or undoped polymer comprising repeat units of formula (I).

A charge-transporting layer or charge-blocking layer may be crosslinked, particularly if a layer overlying that charge-transporting or charge-blocking layer is deposited from a solution. The crosslinkable group used for this crosslinking may be a crosslinkable group comprising a reactive double bond such and a vinyl or acrylate group, or a benzocyclobutane group.

If present, a hole transporting layer located between the anode and the light-emitting layers preferably has a HOMO level of less than or equal to 5.5 eV, more preferably around 4.8-5.5 eV or 5.1-5.3 eV as measured by cyclic voltammetry. The HOMO level of the hole transport layer may be selected so as to be within 0.2 eV, optionally within 0.1 eV, of an adjacent layer (such as a light-emitting layer) in order to provide a small barrier to hole transport between these layers.

If present, an electron transporting layer located between the light-emitting layers and cathode preferably has a LUMO level of around 2.5-3.5 eV as measured by cyclic voltammetry. For example, a layer of a silicon monoxide or silicon dioxide or other thin dielectric layer having thickness in the range of 0.2-2 nm may be provided between the light-emitting layer nearest the cathode and the cathode. HOMO and LUMO levels may be measured using cyclic voltammetry.

A hole transporting layer may contain a homopolymer or copolymer comprising a repeat unit of formula (VII) as described above, for example a copolymer comprising one or more amine repeat units of formula (VII) and one or more arylene repeat units, for example one or more arylene repeat units selected from formulae (III), (IV) and (V).

An electron transporting layer may contain a polymer comprising a chain of optionally substituted arylene repeat units, such as a chain of fluorene repeat units.

If a hole- or electron-transporting layer is adjacent a light-emitting layer containing a phosphorescent material then the T₁ energy level of the material or materials of that layer are preferably higher than that of the phosphorescent emitter in the adjacent light-emitting layer.

Hole Injection Layers

A conductive hole injection layer, which may be formed from a conductive organic or inorganic material, may be provided between the anode 101 and the light-emitting layer 103 of an OLED as illustrated in FIG. 1 to assist hole injection from the anode into the layer or layers of semiconducting polymer. Examples of doped organic hole injection materials include optionally substituted, doped poly(ethylene dioxythiophene) (PEDT), in particular PEDT doped with a charge-balancing polyacid such as polystyrene sulfonate (PSS) as disclosed in EP 0901176 and EP 0947123, polyacrylic acid or a fluorinated sulfonic acid, for example Nafion®; polyaniline as disclosed in U.S. Pat. No. 5,723,873 and U.S. Pat. No. 5,798,170; and optionally substituted polythiophene or poly(thienothiophene). Examples of conductive inorganic materials include transition metal oxides such as VOx, MoOx and RuOx as disclosed in Journal of Physics D: Applied Physics (1996), 29(11), 2750-2753.

Cathode

The cathode 105 is selected from materials that have a workfunction allowing injection of electrons into the light-emitting layer of the OLED. Other factors influence the selection of the cathode such as the possibility of adverse interactions between the cathode and the light-emitting material. The cathode may consist of a single material such as a layer of aluminium. Alternatively, it may comprise a plurality of conductive materials such as metals, for example a bilayer of a low workfunction material and a high workfunction material such as calcium and aluminium, for example as disclosed in WO 98/10621. The cathode may comprise elemental barium, for example as disclosed in WO 98/57381, Appl. Phys. Lett. 2002, 81(4), 634 and WO 02/84759. The cathode may comprise a thin layer of metal compound, in particular an oxide or fluoride of an alkali or alkali earth metal, between the organic layers of the device and one or more conductive cathode layers to assist electron injection, for example lithium fluoride as disclosed in WO 00/48258; barium fluoride as disclosed in Appl. Phys. Lett. 2001, 79(5), 2001; and barium oxide. In order to provide efficient injection of electrons into the device, the cathode preferably has a workfunction of less than 3.5 eV, more preferably less than 3.2 eV, most preferably less than 3 eV. Work functions of metals can be found in, for example, Michaelson, J. Appl. Phys. 48(11), 4729, 1977.

The cathode may be opaque or transparent. Transparent cathodes are particularly advantageous for active matrix devices because emission through a transparent anode in such devices is at least partially blocked by drive circuitry located underneath the emissive pixels. A transparent cathode comprises a layer of an electron injecting material that is sufficiently thin to be transparent. Typically, the lateral conductivity of this layer will be low as a result of its thinness. In this case, the layer of electron injecting material is used in combination with a thicker layer of transparent conducting material such as indium tin oxide.

It will be appreciated that a transparent cathode device need not have a transparent anode (unless, of course, a fully transparent device is desired), and so the transparent anode used for bottom-emitting devices may be replaced or supplemented with a layer of reflective material such as a layer of aluminium. Examples of transparent cathode devices are disclosed in, for example, GB 2348316.

Encapsulation

Organic electronic devices tend to be sensitive to moisture and oxygen. Accordingly, the substrate preferably has good barrier properties for prevention of ingress of moisture and oxygen into the device. The substrate is commonly glass, however alternative substrates may be used, in particular where flexibility of the device is desirable. For example, the substrate may comprise one or more plastic layers, for example a substrate of alternating plastic and dielectric barrier layers or a laminate of thin glass and plastic.

The device may be encapsulated with an encapsulant (not shown) to prevent ingress of moisture and oxygen. Suitable encapsulants include a sheet of glass, films having suitable barrier properties such as silicon dioxide, silicon monoxide, silicon nitride or alternating stacks of polymer and dielectric or an airtight container. In the case of a transparent cathode device, a transparent encapsulating layer such as silicon monoxide or silicon dioxide may be deposited to micron levels of thickness, although in one preferred embodiment the thickness of such a layer is in the range of 20-300 nm. A getter material for absorption of any atmospheric moisture and/or oxygen that may permeate through the substrate or encapsulant may be disposed between the substrate and the encapsulant.

Formulation Processing

A formulation suitable for forming a charge-transporting or light-emitting layer may be formed from the polymer of the invention, any further components of the layer such as light-emitting dopants, and one or more suitable solvents.

The formulation may be a solution of the polymer and any other components in the one or more solvents, or may be a dispersion in the one or more solvents in which one or more components are not dissolved. Preferably, the formulation is a solution.

Solvents suitable for dissolving semiconducting polymers, particularly polymers comprising alkyl substituents, include benzenes substituted with one or more C₁₋₁₀ alkyl or C₁₋₁₀ alkoxy groups, for example toluene, xylenes and methylanisoles.

Particularly preferred solution deposition techniques including printing and coating techniques such spin-coating and inkjet printing.

Spin-coating is particularly suitable for devices wherein patterning of the light-emitting layer is unnecessary—for example for lighting applications or simple monochrome segmented displays.

Inkjet printing is particularly suitable for high information content displays, in particular full colour displays. A device may be inkjet printed by providing a patterned layer over the first electrode and defining wells for printing of one colour (in the case of a monochrome device) or multiple colours (in the case of a multicolour, in particular full colour device). The patterned layer is typically a layer of photoresist that is patterned to define wells as described in, for example, EP 0880303.

As an alternative to wells, the ink may be printed into channels defined within a patterned layer. In particular, the photoresist may be patterned to form channels which, unlike wells, extend over a plurality of pixels and which may be closed or open at the channel ends.

Other solution deposition techniques include dip-coating, roll printing and screen printing.

### EXAMPLES

### Monomer Example 1

Monomer Example 1 was prepared according to the following reaction scheme:

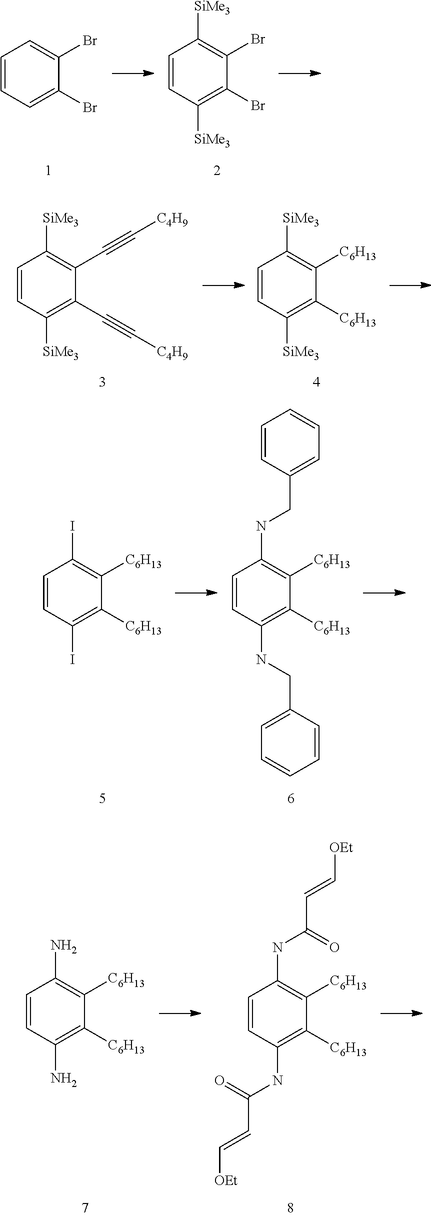

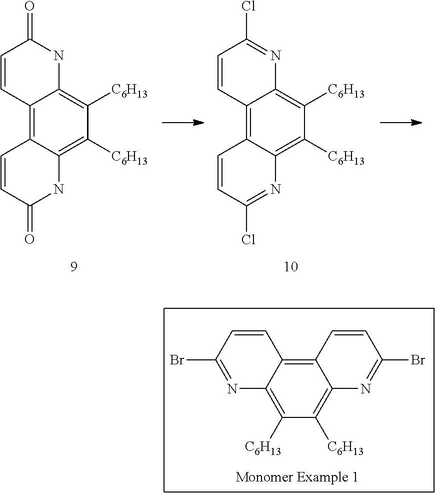

### Intermediate 2

n-BuLi (644.0 mL, 1.61 mol) was added to a solution of diisopropylamine (171.4 g, 1.70 mol) in THF (400 mL) at −25° C. and the mixture stirred for 1 hr and added dropwise to a solution of 1,2-dibromobenzene (100 g, 0.42 mol) and trimethylsilyl chloride (184.1 g, 1.70 mol) in THF (500 mL) at −78° C. using dry ice/acetone bath. After stirring at this temperature for 3 hr the reaction mixture was warmed −10° C., quenched with 1.5N HCl (625 mL), warmed to RT and extracted with EtOAc (1 L). The combined organic layers were dried, concentrated and purified by column chromatography (silica gel, 230-400 mesh, 100% hexane) to yield the title compound (92 g, 57%).

¹H-NMR (400 MHz, CDCl₃): δₚₚₘ 0.40 (s, 18H), 7.34 (s, 2H)

MS: 380 (M⁺); HPLC: 57%

### Intermediate 3

A mixture of intermediate 2 (100 g, 0.26 mol), copper iodide (10.0 g, 0.05 mol) and piperidine (2500 mL) were placed in an autoclave. After purging with N₂ gas for 15 min, Pd(PPh₃)₄ (24.31 g, 0.02 mol) and 1-hexyne (217.0 g, 2.63 mol) were added and the reaction heated at 80° C. for 30 hr. After this time, the piperidine was removed and the residue added to water and extracted with EtOAc (500 mL×3). The combined organic layers were dried, concentrated and purified by Combi-flash chromatography (reverse phase C18 gold column, 100% MeCN) to yield the title compound (24 g, 24%).

¹H-NMR (400 MHz, CDCl₃): δₚₚₘ 0.35 (s, 18H), 0.96 (t, J=7.16 Hz, 6H), 1.52-1.57 (m, 4H), 1.63-1.67 (m, 4H), 2.51 (t, J=7.12 Hz, 4H), 7.34 (s, 2H).

HPLC: 97.3%.

### Intermediate 4

Pd/C (10% w/w) (5.3 g) was added to a solution of intermediate 3 (53.0 g, 0.14 mol) in a mixture of THF/methanol (1:1, 1 L) and hydrogenated at 80° C. at 10 Kg/cm² H₂ pressure for 48 hr. After this time, crude GCMS showed 20% desired 4 and 78% of the partially reduced intermediate. The reaction mixture was filtered through celite and concentrated and the hydrogenation was repeated. After 48 hr, the catalyst was filtered and the reaction mixture concentrated to give the title compound (15 g) which was used in the next step without further purification.

### Intermediate 5

Iodine monochloride (13.74 g, 0.08 mol) was added to a solution of intermediate 4 (15 g, 0.04 mol) in CCl₄ (225 mL) at 0° C. After stirring at 25° C. for 16 hr, the reaction mixture was washed with water (200 mL×2), the organic layer was concentrated and purified by column chromatography (silica gel, 230-400 mesh, 100% hexane) to give the title compound (18 g, 94%).

MS: 498 (M⁺)

### Intermediate 6

Pd(dba)₂ (1.04 g, 1.8 mmol) was added to a N₂ purged solution of intermediate 5 (18.0 g, 0.04 mol), benzyl amine (19.3 g, 0.18 mol), sodium tert-butoxide (14 g, 0.14 mol), rac-BINAP (2.242 g, 3.6 mmol) in toluene (360 mL). After heating at 80° C. for 4 hr, the reaction mixture was cooled to RT and filtered through celite. The filtrate was washed with water (200 mL×2), dried, concentrated and purified by column chromatography (silica gel, 230-400 mesh, 0.5% EtOAc in hexane) to yield the title compound (11 g, 67%).

¹H-NMR (300 MHz, CDCl₃): δₚₚₘ 0.87 (t, J=6.69 Hz, 6H), 1.26-1.32 (m, 12H), 1.45-1.52 (m, 4H), 2.54 (t, J=7.29 Hz, 4H), 4.31 (s, 4H), 6.53 (s, 2H), 7.26-7.41 (m, 10H) MS: 456 (M⁺)

### Intermediate 7

Pd/C (10% w/w) (1.1 g) was added to a solution of intermediate 6 (11 g, 0.02 mol) in a mixture of THF/methanol (300 mL/11 mL) and hydrogenated at 60° C. at 4 Kg/cm² H₂ pressure for 16 hr. After this time, the reaction mixture was filtered through celite and concentrated. The residue was dissolved in EtOAc (500 mL) washed with 10% aqueous NaHCO₃ (10%, 200 mL), dried and concentrated to yield the title compound (6.1 g, 90%).

¹H-NMR (400 MHz, CDCl₃): δₚₚₘ 0.92 (t, J=7.04 Hz, 6H), 1.35-1.39 (m, 8H), 1.41-1.54 (m, 8H), 2.49-2.51 (m, 4H), 2.92 (br, s, 4H), 6.50 (s, 2H)

MS: 276 (M⁺)

### Intermediate 8

Ethoxyacryloyl chloride (8.91 g, 0.07 mol) was added to a solution of intermediate 7 (6.1 g, 0.02 mol) in pyridine (120 mL) at 0° C. After stirring at 25° C. for 16 hr, the reaction mixture was diluted with EtOAc (200 mL), washed with water (100 mL), NaCl (50 mL×2), dried and concentrated to yield the title compound (12 g) which was used in the next step without further purification.

### Intermediate 9

A mixture of Intermediate 8 (12 g) and methane sulphonic acid (120 mL) was heated at 100° C. for 4 hr. After this time the reaction mixture was cooled to RT and poured into an ice-water (1 L) mixture. It was then extracted with EtOAc (500 mL×3), dried and concentrated to yield the title compound (9.3 g) which was used in the next step without further purification.

### Intermediate 10

A mixture of Intermediate 9 (9.3 g) and phosphorous oxychloride (93 mL) was heated at 100° C. for 6 hr. After this time, the phosphorous oxychloride was removed and the residue added slowly to an ice-water (100 mL) mixture and extracted with EtOAc (250 mL×2). The combined organic layers were dried, concentrated and purified by column chromatography (silica gel, 230-400 mesh, 0.5% EtOAc/hexane) to yield the title compound (3.2 g, 31%).

¹H-NMR (400 MHz, CDCl₃): δₚₚₘ 0.94 (t, J=7.00 Hz, 6H), 1.35-1.48 (m, 8H), 1.53-1.60 (m, 4H), 1.66-1.72 (m, 4H), 3.36 (t, J=7.84 Hz, 4H), 7.54 (d, J=8.56 Hz, 2H), 8.72 (d, J=8.64 Hz, 2H)

¹³C-NMR (100 MHz, CDCl₃): δₚₚₘ 14.14, 22.63, 28.25, 29.83, 30.61, 31.62, 121.80, 121.85, 133.23, 141.75, 147.05, 150.48

MS: 417 (M⁺), HPLC: 98.6%

### Monomer Example 1

A mixture of Intermediate 10 (3.2 g, 7.6 mmol) and TMSBr (9.4 g) in propionitrile (40 mL) was heated at 100° C. After 20 hr, the propionitrile was removed and the crude product purified by column chromatography (silica gel, 230-400 mesh, 0.5% EtOAc/hexane) and recrystallized from MeOH (50 mL) to yield the title compound (2.5 g, 31%) as a white solid.

¹H-NMR (400 MHz, CDCl₃): δₚₚₘ 0.94 (t, J=6.88 Hz, 6H), 1.35-1.45 (m, 8H), 1.50-1.56 (m, 4H), 1.65-1.72 (m, 4H), 3.35 (t, J=7.84 Hz, 4H), 7.66 (d, J=8.52 Hz, 2H), 8.60 (d, J=8.60 Hz, 2H)

¹³C-NMR (100 MHz, CDCl₃): δₚₚₘ 14.12, 22.60, 28.29, 29.79, 30.57, 31.58, 122.16, 125.48, 132.72, 141.57, 141.79, 147.67

MS: 506 (M⁺), HPLC: 99.8%

### Polymer Example

A homopolymer was formed by Yamamoto polymerisation of Monomer Example 1.

Modelling Results

Properties of the following polymers were modeled, and results are set out in Table 1:

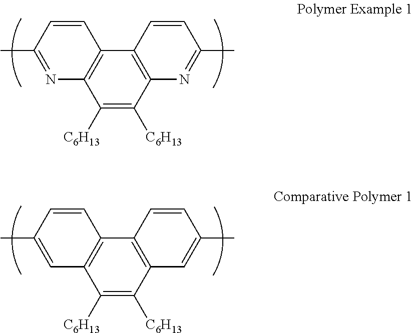

TABLE 1

| HOMOPOLYMER | HOMO/eV | LUMO/eV |
|---|---|---|
| Comparative Polymer 1 | −7.19 | −0.46 |
| Polymer Example 1 | −7.59 | −0.84 |

HOMO and LUMO levels were modeled using AM1 from the AMPAC software package and ZINDO semi-empirical method using Gaussian software.

The LUMO of Polymer Example 1 is significantly lower than that of Comparative Polymer 1 (the skilled person will understand that the absolute values calculated using this method do not necessarily correspond to values obtained by experimental measurement, such as cyclic voltammetry, but measured LUMO values can be expected to reflect the change in LUMO values indicated by this modeling method.

### Devices Example 1

An organic light-emitting device having the following structure was prepared:

ITO/HIL/HTL/LEL/Cathode

wherein ITO is an indium-tin oxide anode; HIL is a hole-injecting layer comprising a hole-injecting material, HTL is a hole-transporting layer, and LEL is a light-emitting layer.

A substrate carrying ITO was cleaned using UV/Ozone. A hole injection layer was formed to a thickness of about 35 nm by spin-coating an aqueous formulation of a hole-injection material. A hole transporting layer was formed to a thickness of about 22 nm by spin-coating a crosslinkable hole-transporting polymer of fluorene repeat units of formula (IV) and arylamine repeat units of formula (VII) and crosslinking the polymer by heating. A light-emitting layer was formed by depositing Polymer Example 1 and a light-emitting dopant to a thickness of about 75 nm by spin-coating. A cathode was formed by evaporation of a first layer of a sodium fluoride to a thickness of about 2 nm, a second layer of aluminium to a thickness of about 100 nm and a third layer of silver to a thickness of about 100 nm.

Although the present invention has been described in terms of specific exemplary embodiments, it will be appreciated that various modifications, alterations and/or combinations of features disclosed herein will be apparent to those skilled in the art without departing from the scope of the invention as set forth in the following claims.

1. A polymer comprising repeat units of formula (I): 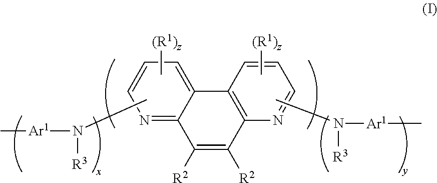
wherein:
R¹ independently in each occurrence is a substituent, and two substituents R¹ may be linked to form a ring;
R² in each occurrence independently is H or a substituent, and two substituents R² may be linked to form a ring;
R³ independently in each occurrence is a substituent;
Ar¹ independently in each occurrence is an aryl or heteroaryl group that may be unsubstituted or substituted with one or more substituents;
x is 0, 1 or 2;
y is 0, 1 or 2; and
z independently in each occurrence is 0, 1 or 2.

2. The polymer according to claim h wherein the repeat unit of formula (I) has formula (Ia):

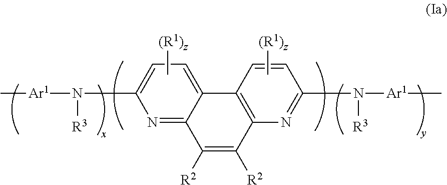

3. The polymer according to claim 1, wherein R² in each occurrence is independently selected from H and C₁₋₄₀ hydrocarbyl.

4. The polymer according to claim 1, wherein each z is 0, and/or x and y are each 0, and/or at least one of x and y is not 0.

5. (canceled)

6. (canceled)

7. The polymer according to claim 1, wherein each Ar¹ is phenyl that may independently in each occurrence be unsubstituted or substituted with one or more substituents.

8. The polymer according to claim 7, wherein the repeat unit of formula (I) has formula (Ib): 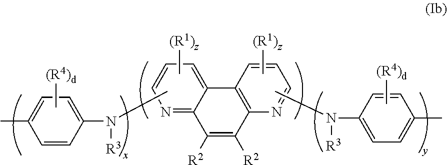
wherein R⁴ independently in each occurrence is a substituent and d independently in each occurrence is 0, 1, 2, 3 or 4.

9. The polymer according to claim 1, wherein R³ independently in each occurrence is an aryl or heteroaryl group that may be unsubstituted or substituted with one or more substituents.

10. The polymer according to claim 9, wherein R³ is phenyl that may be unsubstituted or substituted with one or more substituents.

11. The polymer according to claim 1, wherein the polymer has a LUMO level in the range of about 2.3-2.7 eV as measured by cyclic voltammetry.

12. The polymer according to claim 1, wherein the polymer comprises one or more co-repeat units.

13. The polymer according to claim 12, wherein the polymer comprises a repeat unit of formula (VII): 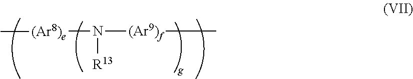
wherein Ar⁸ and Ar⁹ in each occurrence are independently selected from substituted or unsubstituted aryl or heteroaryl, g is greater than or equal to 1, preferably 1 or 2, R¹³ is H or a substituent, preferably a substituent, e and f are each independently 1, 2 or 3; and any two of Ar⁸, Ar⁹ and R¹³ directly linked to the same N atom may be linked by a direct bond or a divalent linking group.

14. The polymer according to claim 12, wherein the polymer comprises one or more repeat units selected from 1,2-phenylene; 1,3-phenylene; 1,4-phenylene; 3,6-fluorene; 2,7-fluorene; indenofluorene; naphthalene; anthracene; phenanthrene; stilbene; pyridine; oxadiazole, triazole; triazine; and imidazole, each of which may be unsubstituted or substituted with one or more substituents.

15. The polymer according to claim 1, wherein the polymer is n-doped.

16. A monomer of formula (Im): 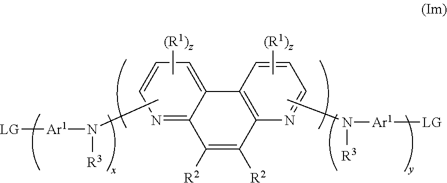
wherein
R¹ independently in each occurrence is a substituent, and two substituents R1 may be linked to form a ring;
R² in each occurrence independently is H or a substituent, and two substituents R2 may be linked to form a ring;
R³ independently in each occurrence is a substituent;
Ar¹ independently in each occurrence is an aryl or heteroaryl group that may be unsubstituted or substituted with one or more substituents;
x is 0, 1 or 2;
y is 0, 1 or 2;
z independently in each occurrence is 0, 1 or 2; and
LG represents a leaving group, with the proviso that if x is zero y is not zero, and if y is zero x is not zero.

17. The monomer according to claim 16, wherein each LG is independently selected from the group consisting of halogens, preferably bromine or iodine;
boronic acids; boronic esters; sulfonic acids; and sulfonic esters.

18. A method of forming a polymer comprising the step of polymerising the monomer according to claim 16.

19. The method according to claim 18, wherein the polymerisation is carried out in the presence of a metal catalyst.

20. A formulation comprising a polymer according to claim 1, and at least one solvent.

21. An organic electronic device comprising an active layer comprising the polymer according to claim 1.

22. (canceled)

23. (canceled)

24. (canceled)

25. (canceled)

26. (canceled)

27. A method of forming an organic electronic device according to claim 21, the method comprising the step of forming the active layer.

28. (canceled)

29. (canceled)
